(19) United States
(12) Patent Application Publication
(10) Pub. No.: US 2021/0301242 A1
(43) Pub. Date: Sep. 30, 2021

(54) SINGLE-USE BIOREACTOR ASSEMBLY WITH INTEGRATED PUMP HEADS

(71) Applicant: Broadley-James Corporation, Irvine, CA (US)

(72) Inventors: Scott T. Broadley, Laguna Beach, CA (US); Robert J. Garrahy, Laguna Niguel, CA (US)

(21) Appl. No.: 17/215,823

(22) Filed: Mar. 29, 2021

Related U.S. Application Data
(60) Provisional application No. 63/001,112, filed on Mar. 27, 2020.

(51) Int. Cl. C12M 1/00

(52) U.S. Cl. CPC C12M 29/18; C12M 23/28; C12M 21/00

(57) ABSTRACT

A bioreactor assembly component can include a length of tubing having an integrated pump head along its length, with a portion of the tubing extending between the pump head and a container, such as a single-use bioreactor. The integrated pump head can be configured to engage with an external driving component to control the operation of the integrated pump head without breaching the sterility of the integrated pump head. The integrated pump head can include only components which can be gamma sterilized, allowing the bioreactor assembly component to be sterilized and stored before use. The bioreactor assembly component can be used with a control module including a pump driver configured to engage with and control the operation of the integrated pump driver, and can include components which can monitor and control the operation of a bioprocess within a single-use bioreactor.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

This application claims the benefit under 35 U.S.C. § 119(e) of U.S. Provisional Application No. 63/001,112, filed Mar. 27, 2020, entitled SINGLE-USE BIOREACTOR ASSEMBLY WITH INTEGRATED PUMP HEADS, the disclosure of which is hereby incorporated by reference in its entirety.

### BACKGROUND

### Technical Field

Embodiments described herein relate to bioreactors, particularly single-use bioreactors.

### Related Art

In many systems, peristaltic pumps are used in conjunction with a control system to move fluid through a length of flexible tubing, and into or out of a bioreactor. A length of tubing may be installed into a peristaltic pump module without disconnecting a length of tubing extending between a bioreactor and another component, maintaining the sterility of the system. A peristaltic pump may be driven, for example, by a brushless motor, and may use rollers outside the tubing to push fluid through the tubing. Each revolution of the rollers of a peristaltic pump causes a known and consistent amount of fluid to flow through the tube connected to the pump.

However, the installation of tubing into peristaltic pumps can be time and labor intensive, and represents a potential point of failure. If the tubing is incorrectly installed, the actual amount of fluid pumped per revolution of the peristaltic pump rotors may differ from the intended amount. This can significantly affect the downstream bioprocess, and can result in a decreased yield or even complete failure of the bioprocess.

In addition, the operation of the peristaltic pump causes damage to the interior of the tubing, due to the cyclical compression of the tubing during operation of the peristaltic pump. For long-term installations, this can result in spalling of the tubing material. This releases small particles of the tubing material into the process flow. These particles can clog downstream components such as filters, and alter the expected operation of those components, or otherwise result in failure of such components. Furthermore, the operation of peristaltic pumps means that the flow is pulsed, rather than constant, resulting in cyclical pressure changes both upstream and downstream of the peristaltic pump.

### SUMMARY

In one broad aspect, a bioreactor assembly is provided, including a single-use bioreactor assembly, the single-use bioreactor assembly including a single-use bioreactor, and a plurality of lengths of tubing extending from the single-use bioreactor, each of the plurality of lengths of tubing including an integrated pump head, and a control module, the control module including a well dimensioned to receive the single-use bioreactor of a single-use bioreactor assembly, and a plurality of pump drivers, each of the plurality of pump drivers configured to engage a sterile integral pump head of the single-use bioreactor assembly to control the operation of the integrated pump head without breaching the sterility of the sterile integral pump head.

The bioreactor assembly can additionally include an agitator disposed within the interior of the single-use bioreactor. The bioreactor assembly can additionally include a plurality of sterile access ports, each of the sterile access ports including an integrated filter. The bioreactor assembly can additionally include at least one of a dissolved oxygen sensor patch located within the single-use bioreactor, the dissolved oxygen sensor patch configured to be readable through a wall of the single-use bioreactor, and a gas well located within a wall of the single-use bioreactor, the gas wall including a gas-permeable membrane The bioreactor assembly can additionally include a pH sensor, the pH sensor including a pH electrode extending into the single-use bioreactor.

The integrated pump head can in some embodiments not include circuitry for controlling the integrated pump head. The integrated pump head can be configured to mechanically couple to an external driving component of one of the plurality of pump drivers. The integrated pump head can be configured to magnetically couple to an external driving component of one of the plurality of pump drivers. The bioreactor assembly can additionally include a plurality of feed reservoirs, each of the feed reservoirs connected to the bioreactor by one of the plurality of lengths of tubing

The control module can additionally include a plurality of mass flow controllers, each of the plurality of mass flow controllers configured to be operably connected to a sterile input port of the single-use bioreactor assembly. The control module can additionally include an agitator connector, the agitator connector configured to be operably connected to an agitator within the single-use bioreactor assembly. The control module can additionally include a pH sensor connector, the pH sensor connector configured to be operably connected to a pH electrode within the single-use bioreactor assembly. The control module can additionally include a temperature control element in thermal communication with a wall of the well. The control module can additionally include a dissolved oxygen sensor configured to read an optical dissolved oxygen patch installed within the single-use bioreactor through a wall of the single-use bioreactor.

The control module can additionally include a support tray for supporting a plurality of feed reservoirs of the single-use bioreactor assembly, each of the feed reservoirs connected to the single-use bioreactor by a length of tubing including one of the plurality of sterile integral pump heads.

In another broad aspect, a single-use bioreactor assembly is provided, including a single-use bioreactor, and a plurality of lengths of tubing extending from the single-use bioreactor, each of the plurality of lengths of tubing including an integrated pump head, the integrated pump head configured to engage with an external driving component to control the operation of the integrated pump head without breaching the sterility of the integrated pump head.

The single-use bioreactor assembly can additionally include an agitator disposed within the interior of the single-use bioreactor. The single-use bioreactor assembly can additionally include a plurality of sterile access ports, each of the sterile access ports including an integrated filter. The single-use bioreactor assembly can additionally include a dissolved oxygen sensor patch located within the single-use bioreactor, the dissolved oxygen sensor patch configured to be readable through a wall of the single-use bioreactor. The single-use bioreactor assembly can additionally include a gas well located within a wall of the single-use bioreactor, the gas wall including a gas-permeable membrane. The single-use bioreactor assembly can additionally include a pH sensor, the pH sensor including a pH electrode extending into the single-use bioreactor.

The integrated pump head can in some embodiments not include circuitry for controlling the integrated pump head. The integrated pump head can be configured to mechanically couple to an external driving component. The integrated pump head can be configured to magnetically couple to an external driving component. The single-use bioreactor can additionally include a plurality of feed reservoirs, each of the feed reservoirs connected to the bioreactor by one of the plurality of lengths of tubing.

The single-use bioreactor assembly can be contained within a sterile package encapsulating the single-use bioreactor assembly. The single-use bioreactor assembly can additionally include a plurality of feed reservoirs, each of the feed reservoirs connected to the bioreactor by one of the plurality of lengths of tubing, and a support frame, the support frame including a first aperture, a second aperture, and a support surface, where the single-use bioreactor is supported within the first aperture, at least one of a plurality of feed reservoirs is supported within the second aperture, and the plurality of pump heads are supported by the support surface. The sterile package can include a first bag encapsulating the single-use bioreactor assembly and a second bag encapsulating the first bag.

In another broad aspect, a control module for use with a single-use bioreactor assembly is provided, the control module including a well dimensioned to receive a single-use bioreactor of a single-use bioreactor assembly, and a plurality of pump drivers, each of the plurality of pump drivers configured to engage a sterile integral pump head of the single-use bioreactor assembly without breaching the sterility of the sterile integral pump head.

The control module can additionally include a plurality of mass flow controllers, each of the plurality of mass flow controllers configured to be operably connected to a sterile input port of the single-use bioreactor assembly. The control module can additionally include an agitator connector, the agitator connector configured to be operably connected to an agitator within the single-use bioreactor assembly. The control module can additionally include a pH sensor connector, the pH sensor connector configured to be operably connected to a pH electrode within the single-use bioreactor assembly. The control module can additionally include a temperature control element in thermal communication with a wall of the well. The control module can additionally include a dissolved oxygen sensor configured to read an optical dissolved oxygen patch installed within the single-use bioreactor through a wall of the single-use bioreactor.

The control module can additionally include a support tray for supporting a plurality of feed reservoirs of the single-use bioreactor assembly, each of the feed reservoirs connected to the single-use bioreactor by a length of tubing including one of the plurality of sterile integral pump heads.

In another broad aspect, a single-use bioreactor assembly component is provided, including a container, a length of tubing extending from the feed reactor, an integral pump head, the integral pump head configured to engage with an external driving component to control the operation of the integrated pump head without breaching the sterility of the integrated pump head, and a sterile connector, where the length of tubing extends between the container and the sterile connector, and where the length of tubing and the integral pump head provide a sterile pathway for a medium to flow between the container and the sterile connector.

The container can include a single-use bioreactor. The sterile connector can be configured to be placed in fluid communication with a feed reservoir to provide a sterile pathway for a supply medium to flow from the feed reservoir into the single-use bioreactor.

The container can include a feed reservoir. The sterile connector can be configured to be placed in fluid communication with a feed reservoir to provide a sterile pathway for a supply medium to flow from the feed reservoir into the single-use bioreactor. The feed reservoir can include a removable lid to allow the feed reservoir to be filled with a supply medium. The removable lid can include a filter to allow air to replace dispensed supply medium.

### BRIEF DESCRIPTION OF THE DRAWINGS

FIG. 1 is a perspective view of a single-use bioreactor assembly including integrated pump heads.

FIG. 2 is a perspective view of the single-use bioreactor assembly of FIG. 1, shown packaged with a temporary support.

FIGS. 3A to 3F illustrate various views of embodiments of control modules configured for use with multiple single-use bioreactor assemblies.

FIG. 4 is a schematic illustration of a control module and a single-use bioreactor assembly such as the bioreactor assembly of FIG. 1.

FIG. 5 is a perspective view of two embodiments of single-use bioreactor assemblies, including an alternative design for a control module.

### DETAILED DESCRIPTION

As an alternative to the use of peristaltic pumps, an inline pump head can be used with a corresponding pump driver to provide a pumping mechanism to move fluid through a length of tubing. Because such a pump mechanism can be designed so that many of the complex and expensive components are located within the pump driver, such that the inline pump head can be made comparatively simple. In such an embodiment, disposable, inexpensive pump heads can be used in conjunction with a reusable pump driver.

In some embodiments, single-use bioreactors may be provided as part of an assembly including tubing with integrated inline pump heads. The entire assembly, including both the bioreactor and the tubing with the integrated inline pump heads, may be sterilized and stored until the bioreactor assembly is to be used.

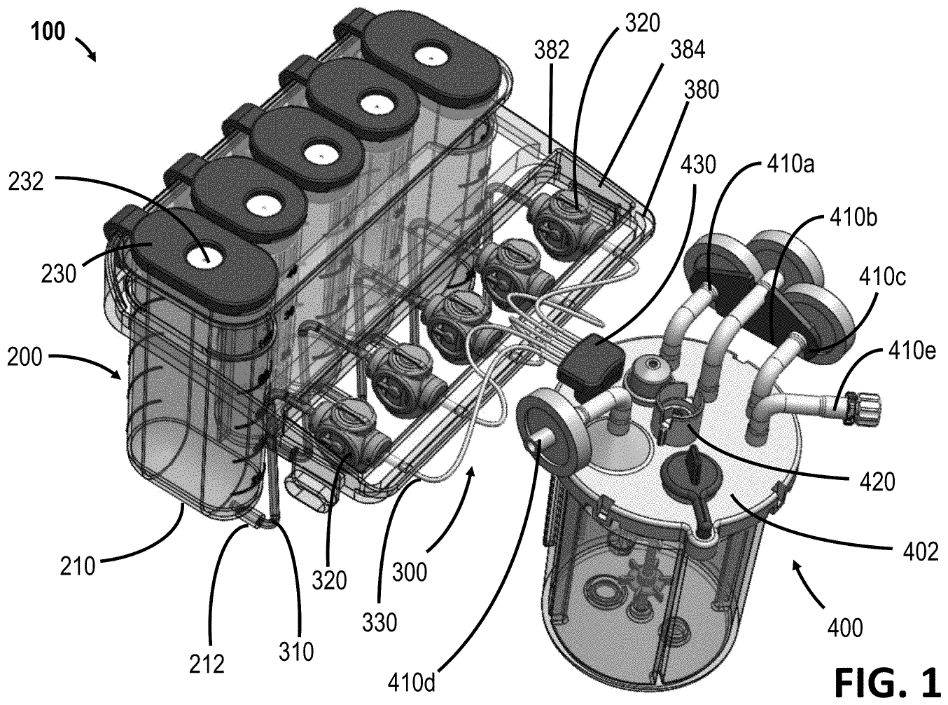

FIG. 1 is a perspective view of a single-use bioreactor assembly including integrated pump heads. The assembly 100 includes a bioreactor 400 which is connected to a plurality of reservoirs 200 by an array 300 of tubing. The bioreactor 400 may be a single-use bioreactor, and may be made primarily from a polymeric material or other suitable material which can withstand gamma sterilization. In the illustrated embodiment, the bioreactor is generally cylindrical in shape, with a plurality of grooves in the outer sidewall to facilitate placement and retention of the bioreactor, although a wide variety of other suitable shapes may be used. In some embodiments, the bioreactor 400 may be as small as 250 mL in volume, or smaller, or may be as large as 2000 L or larger.

The bioreactor 400 can include various sensors, ports, and other components which can be used in configuring the bioreactor 400 for and controlling a variety of bioprocesses. The bioreactor 400 may include a plurality of ports 410a-410e which can be used to introduce or remove gas from the bioreactor 400. In the illustrated embodiment, ports 410a-410d include an integrated filter, while the port 410e does not. These ports can be used, for example, to supply the bioreactor with oxygen, carbon dioxide, nitrogen, and an air mixture, in order to control the amount and type of aeration within the bioreactor 400. In some embodiments, the ports 410a-410e may provide fluid paths to or slightly beyond the interior sidewall of the bioreactor 400, while in other embodiments, certain of the ports 410a-410e may be connected to an internal structure such as a pipe extending from an internal sidewall of the bioreactor 400 to allow delivery of a gas to a specific location within in the bioreactor, such as near the base of the bioreactor. The bioreactor 400 can also include an agitator 422 which can be driven via an external motor mechanically coupled to the agitator 422.

The bioreactor 400 can also include various sensors, in order to monitor and control an ongoing bioprocess. For example, the bioreactor 400 can include sensors configured to measure a pH value, a dissolved oxygen (DO) level, a carbon dioxide (CO₂ level), a temperature or any other suitable measurement. For example, a pH sensor can include a pH electrode 450 located within or extending into the bioreactor 400. A DO sensor can include an optical DO patch installed in the vessel, and configured to be read through a translucent wall or other section of the bioreactor 400. In other embodiments, a gas well can be provided in a wall of the bioreactor 400, and used to measure DO or other parameters such as CO₂ through a gas-permeable membrane, such as a silicone rubber membrane.

Electrical and/or mechanical connection with the various sensors and other components of the bioreactor 400 may be provided via one or more connectors 420. In the illustrated embodiment, a connector 420 is located on the lid 402 of the bioreactor 400. The other components extending into or through the bioreactor 400 are in the illustrated embodiment also extend into or through the lid 402 of the bioreactor 400. As discussed in greater detail below, this arrangement allows the bioreactor 400 to be seated within a control module with the lid 402 generally flush with a surface of the control module. In other embodiments, however, some or all of these components may extend through a sidewall or base of the bioreactor 400.

In the illustrated embodiment, the reservoirs 200 have generally rounded obround cross-sectional areas, and may be polymeric feed bottles, or may be made from another suitable material which can withstand gamma sterilization. In the illustrated embodiment, the reservoirs 200 have a generally obround cross-sectional shape. In the illustrated embodiment, two of the reservoirs 200 are larger in volume than the other three of the reservoirs 200, although in other embodiments any suitable combination of number and sizes of reservoirs may be used.

The reservoirs 200 include lids 230 which can be removed to allow the reservoirs 200 to be filled with media or solutions to be used in a bioprocess. In the illustrated embodiment, the lids 230 of the reservoirs 200 include a filter 232 which can allow air to flow into the reservoirs 200 during use to displace a medium or solution that is pumped out of the reservoirs 200. These reservoirs 200 may be filled, for example, with additional process media, alkaline solution, anti-foam, or any other suitable material. In other embodiments, one or more of the reservoirs 200 may be left empty at the beginning of the bioprocess.

The filling of these reservoirs 200 may be done, for example, within a clean hood or other sterile environment. This may occur once the assembly 100 has been set up and connected to a control module as described in greater detail below, or may be done prior to setting up and connecting the assembly 100 to a control module. In embodiments in which the assembly 100 is used with a larger bioreactor, the size of the reservoirs 200 may mean that the reservoirs are filled elsewhere before being connected to the bioreactor. In such an embodiment, an assembly 100 may be used which does not include the bioreactor, but instead includes tubing extending from the integral pump head which terminates in a sterile connection, such as a sterile hose connector, which can be used to connect to the bioreactor after the reservoirs have been filled. Such embodiments are discussed in greater detail below.

Extending from an outlet port 212 near the bases 210 of the reservoirs 200 is a length of reservoir-side tubing 310. Each length of reservoir-side tubing extends between one of the reservoirs 200 and an integral pump head 320. Extending from the opposite port of each integral pump head 320 is a length of bioreactor-side tubing 330, which enters the bioreactor 400 via a manifold 430.

Because the pump head 320 is configured to be connected to and function in conjunction with a separate pump driver, the pump head 320 may include only a subset of the components required to form a complete pumping mechanism, and in particular may include only components which can withstand gamma sterilization.

In particular, the pump head 320 may not include a driving motor, a power source, or control circuitry, but may instead include mechanical pump components which can be connected to a pump driver. The pump driver can instead include those components missing from the pump head 320 and will interact with the mechanical pump components to move fluid through the pump head 320.

Because the entire assembly 100 can be gamma-sterilized, and the pump heads 320 are integral with the tubing 310 and 330 connecting the reservoirs 200 to the bioreactor 400, the assembly 100 can be connected to pump drivers for each of the pump heads 320 without compromising the sterility of the assembly 100. In addition, because the pump heads 320 can in some embodiments be snapped onto or otherwise easily and securely connected to the pump driver, the possibility for human error in assembling the pumping mechanism can be substantially eliminated.

Because the pump head 320 is integral with the assembly 100, and in particular with a specific reservoir 200, the pump head 320 can be chosen to be suitable for use with that reservoir 200. In an embodiment in which there is substantial variation between the sizes of the various reservoirs 200, different pump heads 320 may be used for different reservoirs. Similarly, a pump head 320 with a smaller pumping capacity may allow greater precision in dosing, and a reservoir 200 and pump head 320 may be selected to provide a reservoir suitable for more precise dosing at a lower overall flow rate.

Because the pump head 320 can be reversible, the pump head 320 can also be used to remove material from the bioreactor 400 during or at the conclusion of the bioprocess. In a bioprocess in which such removal is intended to occur, the pump head 320 may be installed in the opposite direction in line, or the pump heads 320 may be configurable to be driven in reverse via the driving module. In other embodiments, one of the pump heads may be provided with sufficient additional tubing to allow the pump head to be installed onto the pump driver in the opposite orientation. In some embodiments, the reservoir may also include a length of tubing extending from the bioreactor, such as the base of the bioreactor, which can include an integrated pump head and can be used to draw a sample of the process media within the bioreactor

Because the pump heads 320 are part of a system which is not gravity-driven, the pumping components such as the pump heads 320 may be located at a height above the base of the feed reservoirs 200.

The assembly 100 may include a section configured to support the pump heads 320. In the illustrated embodiment, the pump heads 320 are supported by a tray 380 extending laterally outward from the bay of reservoirs 200. In some embodiments, the tray 380 may form part of a structure which extends at least partially around the reservoirs 200 and holds the reservoirs 200 in place.

The tray 380 may include a central aperture 382 in which the pump heads are suspended, with a portion of the reservoir-side tubing 310 passing through or otherwise supported by the inner side of the tray 380 adjacent the reservoirs 200, and the bioreactor-side tubing 330 passing through or otherwise supported by the outer side of the tray 380 located away from the reservoirs 200. A cover 384 may be removed or lifted about a hinge to access the pump heads 320 during installation, and subsequently closed to provide additional protection to the pump heads 320. Suspension of the pump heads 320 between shorter subsections of the reservoir-side tubing 310 and the bioreactor-side tubing 330 can provide sufficient play to allow the pump heads 320 to be snapped onto or otherwise engaged with the driving motors, while ensuring that the pump heads 320 can only be installed on a particular driving motor, reducing the risk of inaccurate configuration of the assembly 100.

In some embodiments, the assembly 100 can be gamma-sterilized, and stored in a sterile manner. In some embodiments, the assembly 100 can be stored within two nested bags to ensure sterility. The bags may comprise a gamma-resistant polymer material and may in some embodiments be partially clear or translucent. The apparatus may then be gamma sterilized to provide a sterilized apparatus within a package that will maintain the sterility for an extended period of time. In some embodiments, an apparatus packaged in this manner may be certified to maintain sterilization for 24 months or longer. When needed, the bags may be opened within a clean hood or inside another sterile environment to be used for a bioprocess.

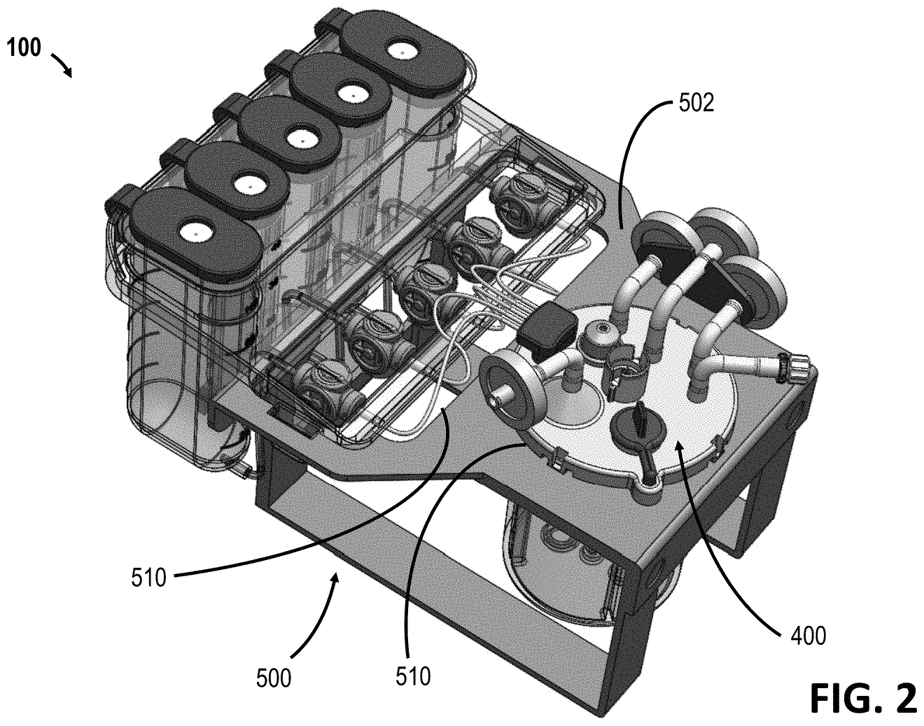

FIG. 2 is a perspective view of the single-use bioreactor assembly of FIG. 1, shown packaged with a temporary support. Because the various components of the assembly 100 are not rigidly coupled to one another, but are instead connected via flexible tubing, a storage support 500 may be provided and used to support the assembly 100 during storage or during or after media filling. As described above, the feed reservoirs may be filled within a clean hood, and then carried over to a control module for installation and use. The storage support 500 provides support during the media filling and handling process prior to installation.

In one exemplary embodiment, the storage support 500 may include an upper surface 502 at substantially the same level as the underside of the tray 380, to provide support to the tray 380 and the bay of reservoirs 200. The upper surface may include a first aperture 510 in the upper surface 502 of the storage support 500, within which a portion of the tray 380 may be seated. In some embodiments, the underside of the tray 380 may be press-fit into the first aperture.

The bioreactor 400, which is connected to the remainder of the apparatus only by lengths of flexible bioreactor-side tubing 330, is separately supported within a second aperture 520 in the upper surface 502 of the storage support 500. The spacing between the first aperture 510 and the second aperture 520 may be chosen to allow slack in the flexible bioreactor-side tubing 330 and to prevent the bioreactor 400 from being pulled away from the remainder of the apparatus, putting strain on the bioreactor-side tubing 330 or dislodging or otherwise damaging portions of the apparatus. In other embodiments, other storage supports or methods of storing the assembly 100 may be utilized. The bioreactor could be clipped directly to a tray or another component of the apparatus, or a removable bridge or base section may be used as an additional or alternative support structure.

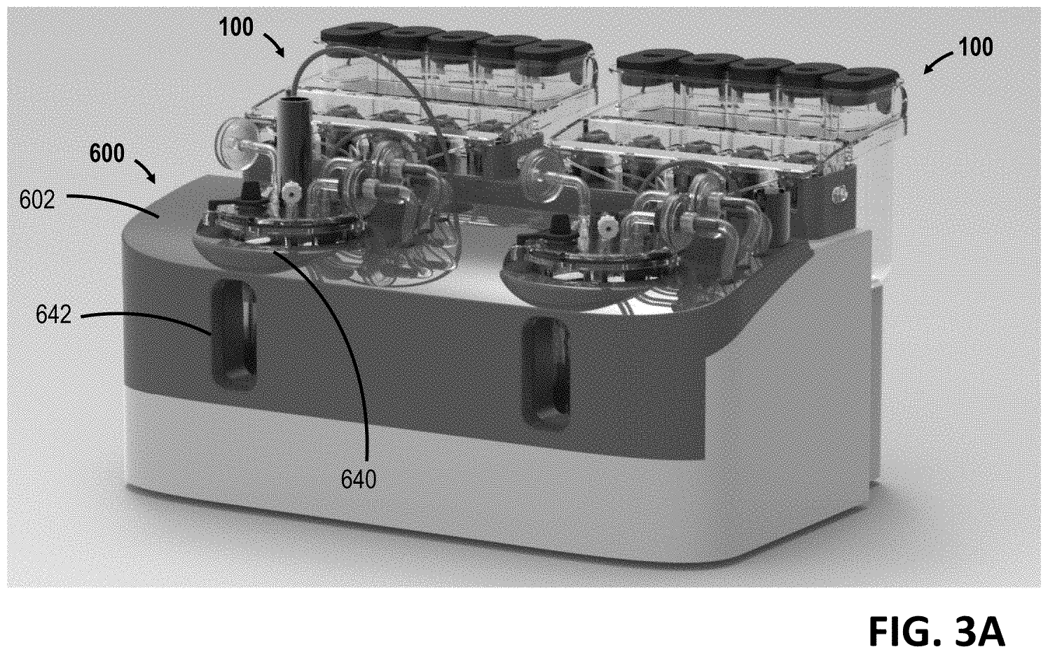

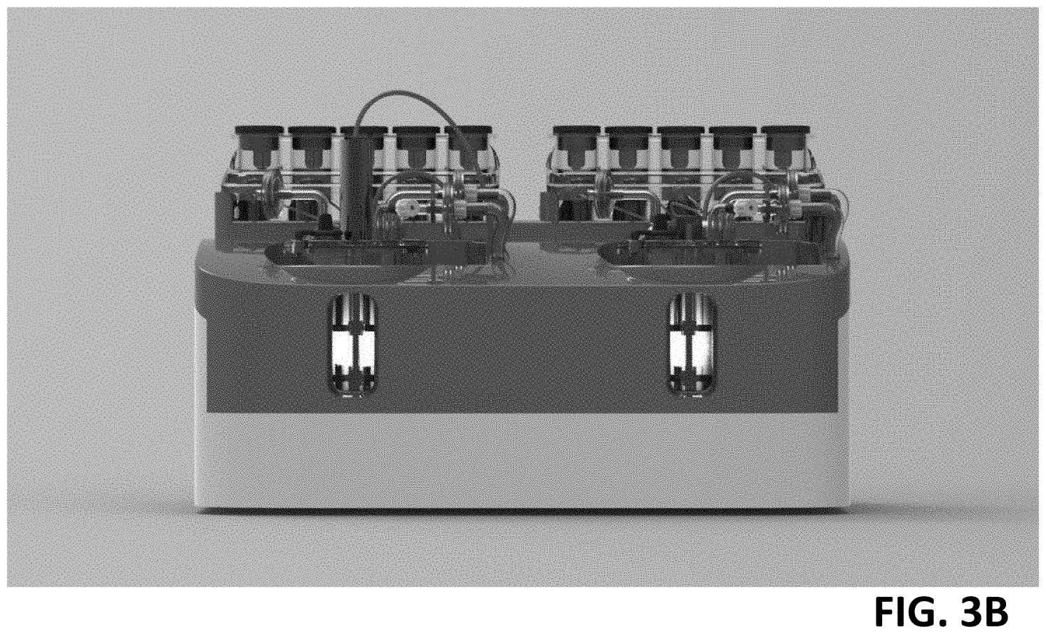

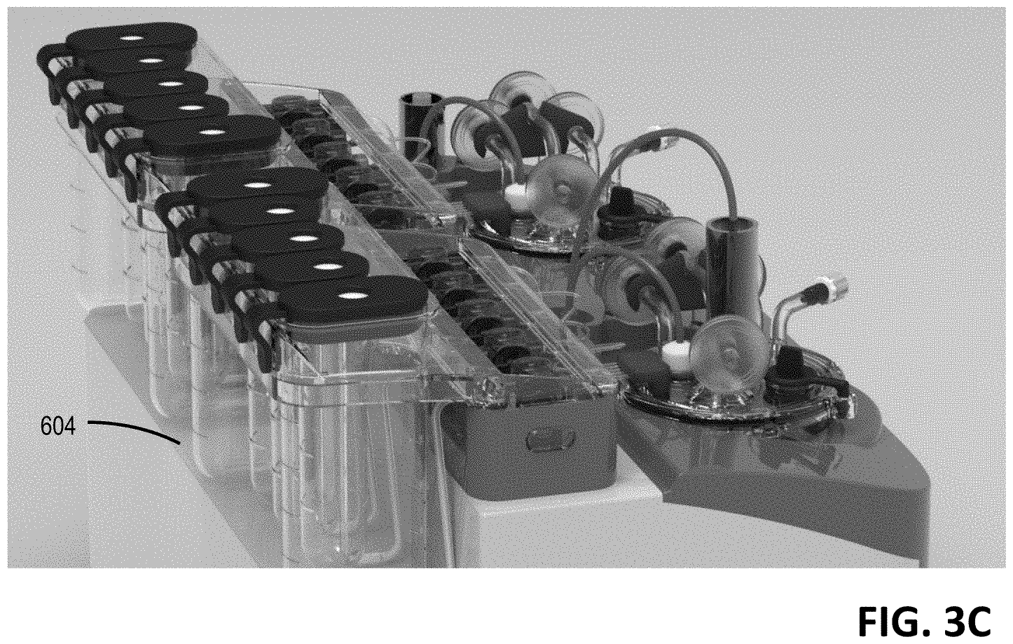

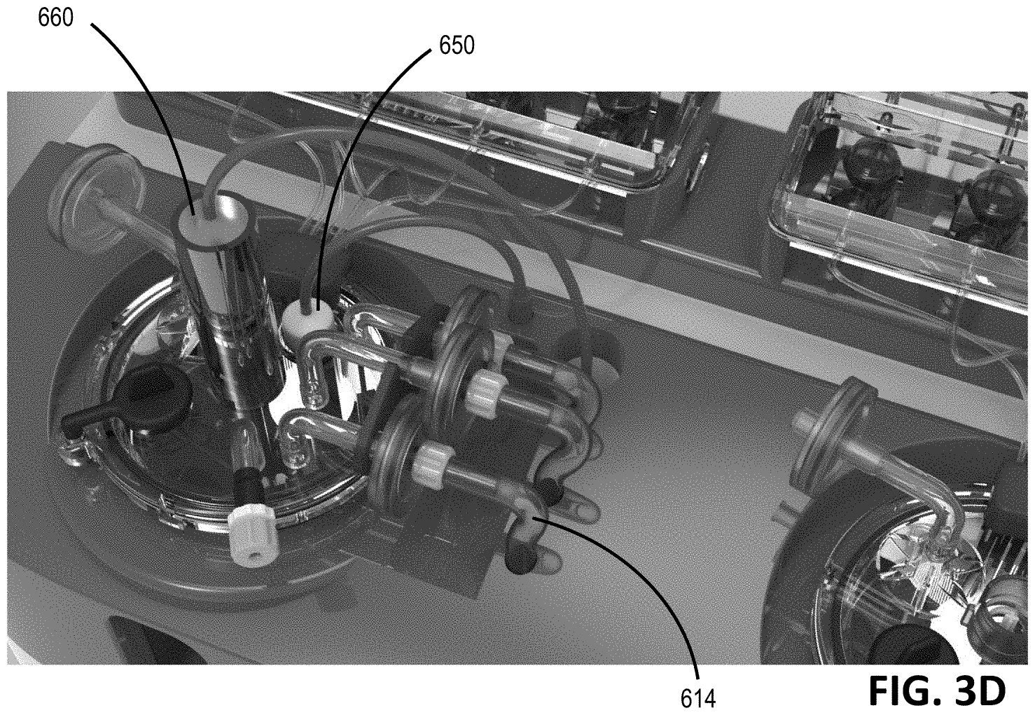

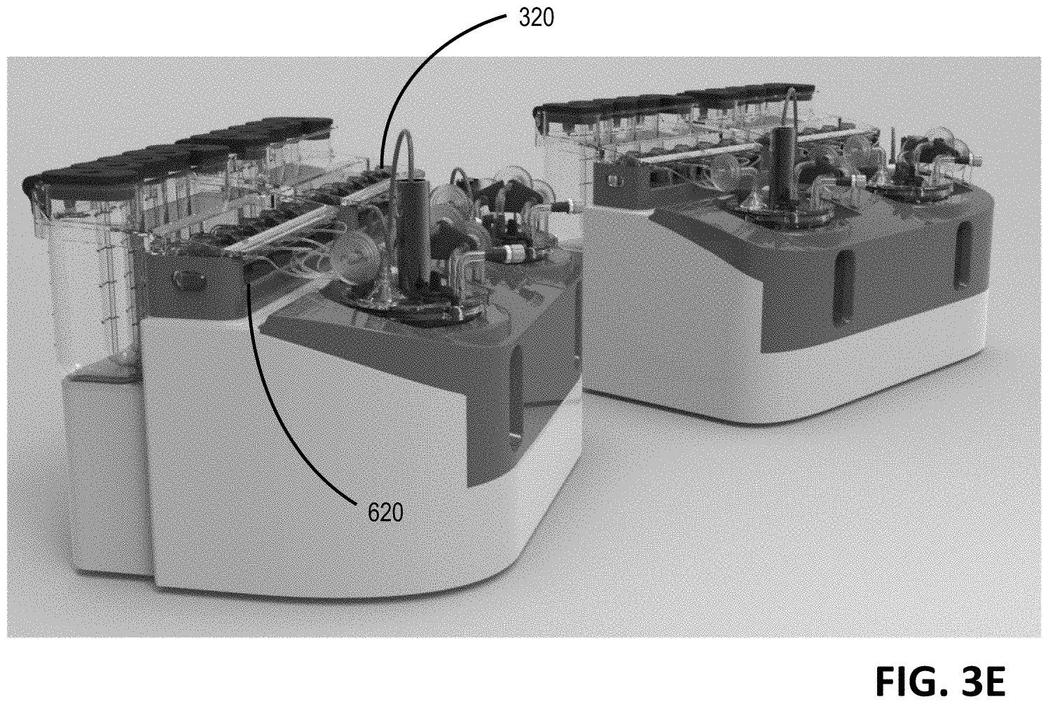

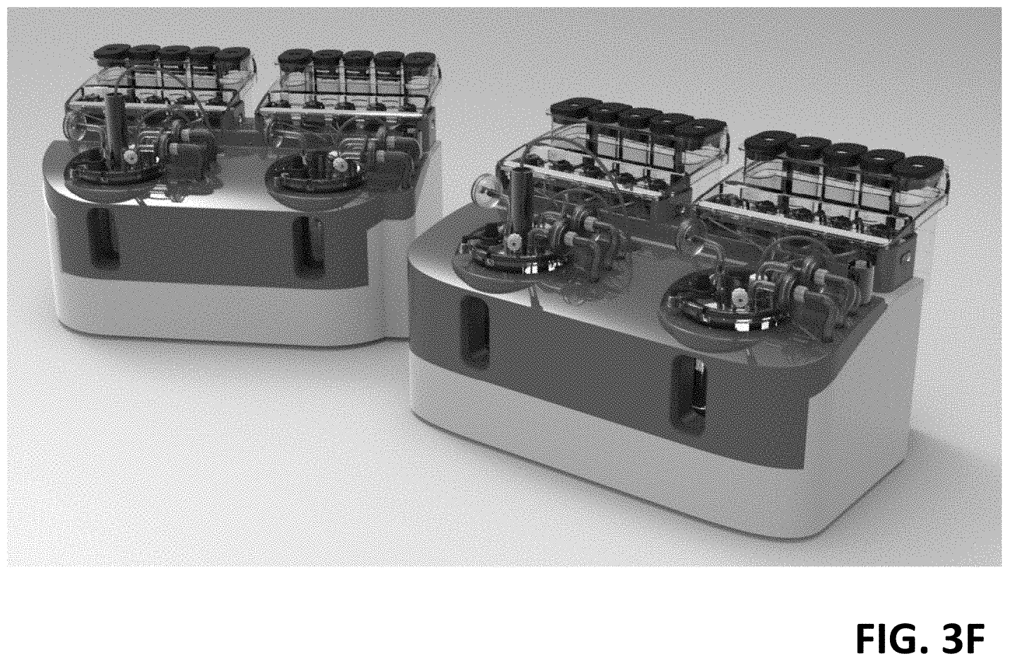

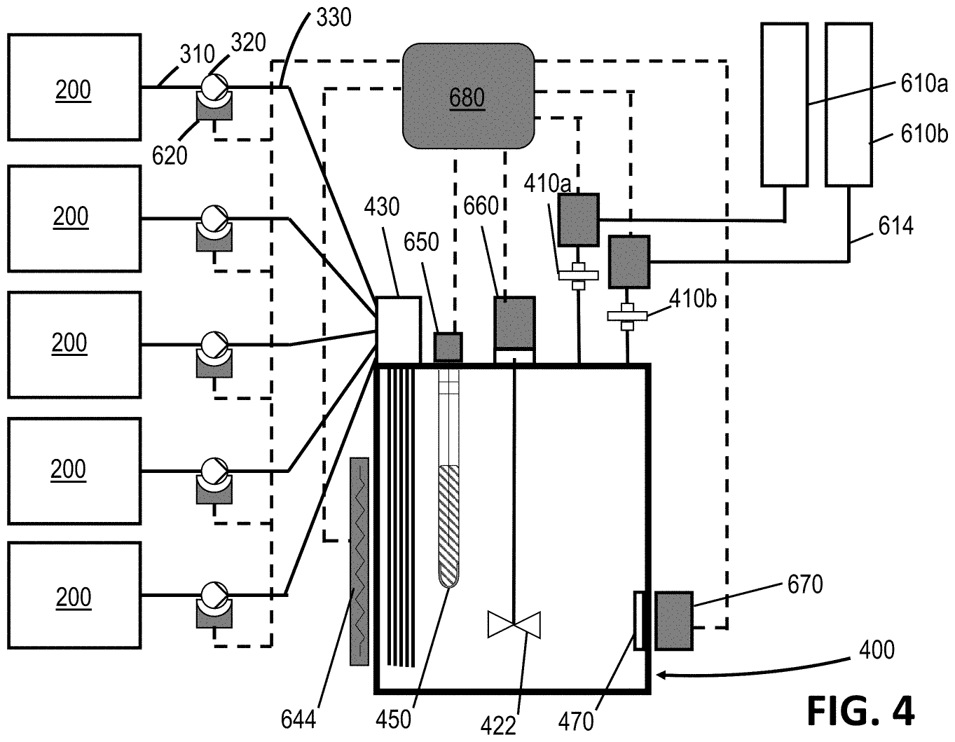

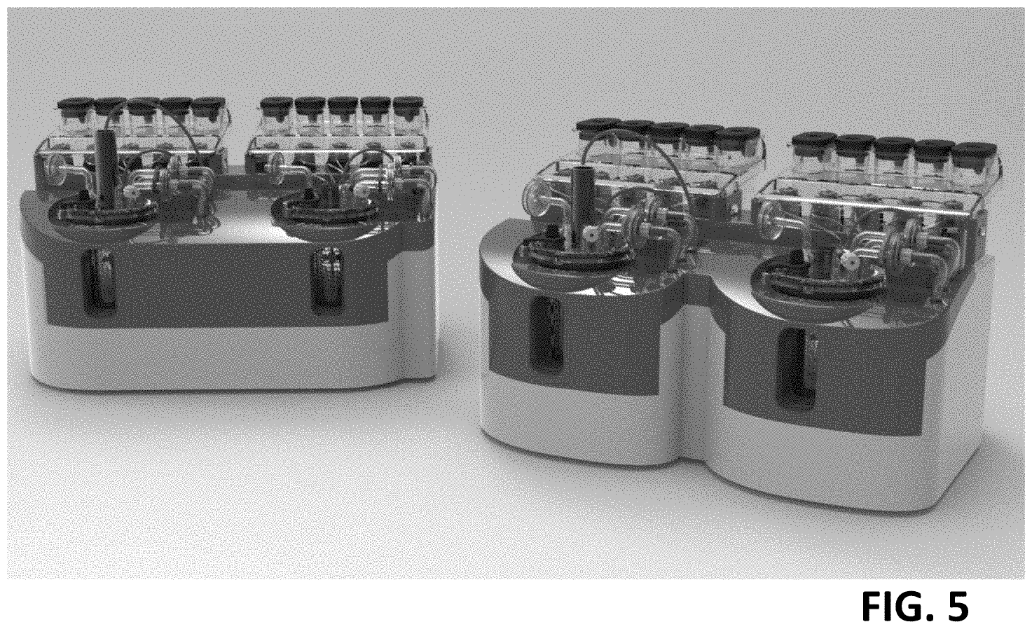

FIGS. 3A to 3F illustrate various views of embodiments of control modules configured for use with multiple single-use bioreactor assemblies. FIG. 4 is a schematic illustration of a control module and a single-use bioreactor assembly such as the bioreactor assembly of FIG. 1. FIG. 5 is a perspective view of two embodiments of single-use bioreactor assemblies, including an alternative design for a control module.

The single-use bioreactor assembly 100 may be configured to be used in conjunction with a reusable control module 600. The control module 600 is configured to be connected to or otherwise interact with various components of the assembly 100 in a sterile manner to control a bioprocess within the bioreactor 400.

In the illustrated embodiment, the control module 600 comprises a contoured housing 602 having features configured to retain and support the various components of the single-use bioreactor assembly 100. In the illustrated embodiment, the control module 600 is configured to retain and control two separate single-use bioreactor apparatuses 100. The tray 380 may be snapped onto the housing 602 by pressing the tray 380 between upwardly extending flanges, such that raised tabs extending laterally outward from the sides of tray 380 will be inserted into the apertures in the flanges of the housing 602. The reservoirs 200 may rest on a shelf 604 extending from the back of the housing 602.

Snapping the tray 380 onto the housing 602 will position the loosely-suspended pump heads 320 over underlying pump drivers 620. The pump drivers 620 are configured to be operably connected to the pump heads 320 to drive the pump heads 320 without breaching the sterility of the in-line pump heads 320. In some embodiments, the pump heads 320 may be snapped onto the pump drivers 620. The loose suspension of the pump heads 320 provides sufficient play to allow the pump heads 320 to be snapped onto or disconnected from the appropriate underlying pump driver 620, but can be designed such that a given pump head 320 is not provided with sufficient slack in the tubing to allow the pump head 320 to be snapped onto a different pump driver 620, such as a pump driver 620 adjacent to the intended pump driver 620.

In some embodiments, the pump drivers 620 include rotatable features configured to mesh with or otherwise engage rotatable components of the pump heads 320. In one embodiment, the pump drivers 620 include one or more stepper motors configured to drive the rotatable features of the pump drivers 620, such that the connection between the pump drivers 620 and the pump heads 320 is purely mechanical. In some embodiments, magnetic coupling may be used to connect the pump drivers 620 and the pump heads 320. The pump drivers 620 may also include or be in electrical connection with driving circuitry configured to control the operation of the stepper motors and other components of the pump drivers. In other embodiments, an electrical connection may be made with the pump heads 320, and one or more stepper motors may be located within the pump heads and be powered by and driven by the pump driver 620.

In some embodiments, the pump driver may be a smart pump driver, and may have information regarding the pump driver stored thereon or therein in a manner which is readable by a user, a control module, or another instrument. For example, the pump driver may include an RFID chip that includes information regarding the pump head. This information may include identifying information, such as a serial number or batch number, and may include operational information, such as calibration data, throughput information, capacity information, or displacement information of the pump.

The bioreactor 400 may be seated within a well 640 formed in the housing 602. In the illustrated embodiment, the well 640 is positioned near the front wall of the housing 602, such that a window 642 formed in the front wall of the housing 602 allows visual monitoring of the bioreactor 400, even when the well 640 is sufficiently deep that the bulk of the bioreactor 400 is located below the upper surface of the housing 602 when seated within the well 640. The bioreactor 400 may then be connected to various components of the control module 600, and placed in sufficiently close proximity to other components that they can be used to affect or control a bioprocess occurring within the bioreactor 400.

In an embodiment in which the bioreactor 400 includes an integrated pH sensor having a pH electrode 450, a pH connector 650 configured to place the pH sensor in electrical communication with the control module 600 can be connected to the pH sensor of the bioreactor 400 once the bioreactor 400 is seated within the well 640. Similarly, an agitator connector 660, which may include or be operably connected to a driving motor configured to drive an agitator 422 of the bioreactor 400, or may provide power and control to a motor included in the bioreactor 400.

Supply tubing 614 which is in fluid communication with mass flow controllers 610a and 610b disposed within or adjacent the control module 600 can be connected to the sterile ports 410a-410e of the bioreactor 400. The mass flow controllers can control the provision of air, nitrogen (N2), oxygen (O2), carbon dioxide (CO2) or other suitable gases or liquids to the bioreactor 400 in a sterile and controlled manner. The mass flow controllers may include flow sensors which monitor the amount of gases or liquids provided by the mass flow controllers. Vent tubing may be connected to the vent port of the bioreactor 400.

The control module 600 may also include additional components disposed within the well 604. One or more temperature control elements 644 may be located within the well 604, such that they will be in contact or close proximity with the bioreactor 400 to provide heating or other temperature control of the bioreactor 400 during, before, or after the bioprocess. A dissolved oxygen (DO) sensor module 670 may be used in conjunction with a DO patch 470 located within the bioreactor 400 to provide an indication of the dissolved oxygen levels within the bioreactor 400.

The control module 600 may include a controller 680. The controller 680 may include some or all of a processor, a memory, and an input/output module such as a wired or wireless communication link. The controller 680 may be configured to control the operation of one or more components of the control module 600 and the bioreactor 400 retained therein, including the pump motors 620, the mass flow controllers 610a and 610b, the DO sensor 670, the pH sensor 450, the agitator 422, and the heating (or other temperature control) element 644. In the illustrated embodiment, the controller 680 is schematically illustrated as a single module. In other embodiments, some or all of the various components of the controller 680 and the bioreactor 400 may be performed by discrete software and/or hardware modules, or by combinations of discrete software and/or hardware modules.

The control module 600, which can include the pump drivers 620, can be reused with a number of single-use assemblies 100 including bioreactors 400 and integrated tubing array 300 including the in-line, integral pump heads 320. By integrating only the pump heads 320 within the single-use assembly 100, the pump drivers 620 can be reused for multiple bioprocesses. However, in other embodiments, additional pump components can be included within the single-use assembly 100. For example, in some embodiments, driving components may be integrated into the single-use assembly 100, and an electrical connection may be made with reusable control circuitry located within the control module 600 or elsewhere.

In some embodiments, particularly embodiments in which the feed reservoirs and/or the single-use bioreactor have a larger capacity, an assembly may be provided which includes tubing with an integrated pump head, where the tubing is connected at one end to one of a feed reservoir or a single-use bioreactor and at the other end to a sterile connector. For example, an assembly may include a feed reservoir connected to a length of tubing, where the tubing includes an integrated pump head and terminates in a sterile connector. Such an assembly may be subsequently connected to a bioreactor such as a single-use bioreactor using the sterile connector to complete the sterile pathway between the feed reservoir and a single use bioreactor. The assembly may include a plurality of feed reservoirs, each connected to a length of tubing with an integrated pump head and terminating in a sterile connector.

Similarly, an assembly may include a single-use bioreactor connected to one or more lengths of tubing, where the tubing includes an integrated pump head and terminates in a sterile connector. The assembly may include a plurality of lengths of tubing, each length of tubing having an integrated pump head and terminating in a sterile connector. Such an assembly may be subsequently connected to one or more feed reservoirs using the sterile connector to complete the sterile pathway between the feed reservoir and a single use bioreactor. In some embodiments, the single-use bioreactor may be a bioreactor bag for use with a rigid vessel.

In the foregoing description, specific details are given to provide a thorough understanding of the examples. However, it will be understood by one of ordinary skill in the art that the examples may be practiced without these specific details. Certain embodiments that are described separately herein can be combined in a single embodiment, and the features described with reference to a given embodiment also can be implemented in multiple embodiments separately or in any suitable subcombination. In some examples, certain structures and techniques may be shown in greater detail than other structures or techniques to further explain the examples.

The previous description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

What is claimed is:

1. A \bioreactor assembly, comprising:
a single-use bioreactor assembly, the single-ise bioreactor assembly comprising:
a single-use bioreactor; and
a plurality of lengths of tubing extending from the single-use bioreactor, each of the plurality of lengths of tubing including an integrated pump head; and
a control module, the control module comprising:
a well dimensioned to receive the single-use bioreactor of a single-use bioreactor assembly; and
a plurality of pump drivers, each of the plurality of pump drivers configured to engage a sterile integral pump head of the single-use bioreactor assembly to control the operation of the integrated pump head without breaching the sterility of the sterile integral pump head.

2. The bioreactor assembly of claim 1, additionally comprising an agitator disposed within the interior of the single-use bioreactor.

3. The bioreactor assembly of claim 1, additionally comprising a plurality of sterile access ports, each of the sterile access ports including an integrated filter.

4. The bioreactor assembly of claim 1, additionally comprising at least one of:
a dissolved oxygen sensor patch located within the single-use bioreactor, the dissolved oxygen sensor patch configured to be readable through a wall of the single-use bioreactor;
a gas well located within a wall of the single-use bioreactor, the gas wall comprising a gas-permeable membrane; and
a pH sensor, the pH sensor including a pH electrode extending into the single-use bioreactor.

5. The bioreactor assembly of claim 1, wherein the integrated pump head does not include circuitry for controlling the integrated pump head.

6. The bioreactor assembly of claim 1, wherein the integrated pump head is configured to mechanically or magnetically couple to an external driving component of one of the plurality of pump drivers.

7. The bioreactor assembly of claim 1, additionally comprising a plurality of feed reservoirs, each of the feed reservoirs connected to the bioreactor by one of the plurality of lengths of tubing.

8. The bioreactor assembly of claim 1, wherein the control module additionally comprises a plurality of mass flow controllers, each of the plurality of mass flow controllers configured to be operably connected to a sterile input port of the single-use bioreactor assembly.

9. The bioreactor assembly of claim 1, wherein the control module additionally comprises at least one of:
an agitator connector, the agitator connector configured to be operably connected to an agitator within the single-use bioreactor assembly;
a pH sensor connector, the pH sensor connector configured to be operably connected to a pH electrode within the single-use bioreactor assembly;
a temperature control element in thermal communication with a wall of the well; and
a dissolved oxygen sensor configured to read an optical dissolved oxygen patch installed within the single-use bioreactor through a wall of the single-use bioreactor.

10. The bioreactor assembly of claim 1, wherein the control module additionally comprises a support tray for supporting a plurality of feed reservoirs of the single-use bioreactor assembly, each of the feed reservoirs connected to the single-use bioreactor by a length of tubing including one of the plurality of sterile integral pump heads.

11. A single-use bioreactor assembly, comprising:
a single-use bioreactor; and
a plurality of lengths of tubing extending from the single-use bioreactor, each of the plurality of lengths of tubing including an integrated pump head, the integrated pump head configured to engage with an external driving component to control the operation of the integrated pump head without breaching the sterility of the integrated pump head.

12. The single-use bioreactor assembly of claim 11, additionally comprising at least one of:
an agitator disposed within the interior of the single-use bioreactor;
a plurality of sterile access ports, each of the sterile access ports including an integrated filter;
a dissolved oxygen sensor patch located within the single-use bioreactor, the dissolved oxygen sensor patch configured to be readable through a wall of the single-use bioreactor;
a gas well located within a wall of the single-use bioreactor, the gas wall comprising a gas-permeable membrane; and
a pH sensor, the pH sensor including a pH electrode extending into the single-use bioreactor.

13. The single-use bioreactor assembly of claim 11, wherein the integrated pump head is configured to mechanically or magnetically couple to an external driving component.

14. The single-use bioreactor assembly of claim 11, wherein the single-use bioreactor assembly is contained within a sterile package encapsulating the single-use bioreactor assembly.

15. The single-use bioreactor assembly of claim 14, additionally comprising
a plurality of feed reservoirs, each of the feed reservoirs connected to the bioreactor by one of the plurality of lengths of tubing; and
a support frame, the support frame including a first aperture, a second aperture, and a support surface, wherein the single-use bioreactor is supported within the first aperture, at least one of a plurality of feed reservoirs is supported within the second aperture, and the plurality of pump heads are supported by the support surface.

16. The sterilized package of claim 14, wherein the sterile package includes a first bag encapsulating the single-use bioreactor assembly and a second bag encapsulating the first bag.

17. A single-use bioreactor assembly component, comprising:
a container; and
a length of tubing extending from the feed reactor;
an integral pump head, the integral pump head configured to engage with an external driving component to control the operation of the integrated pump head without breaching the sterility of the integrated pump head; and
a sterile connector, wherein the length of tubing extends between the container and the sterile connector, and wherein the length of tubing and the integral pump head provide a sterile pathway for a medium to flow between the container and the sterile connector.

18. The component of claim 17, wherein the container comprises a single-use bioreactor, and wherein the sterile connector is configured to be placed in fluid communication with a feed reservoir to provide a sterile pathway for a supply medium to flow from the feed reservoir into the single-use bioreactor.

19. The component of claim 17, wherein the container comprises a feed reservoir, and wherein the sterile connector is configured to be placed in fluid communication with a feed reservoir to provide a sterile pathway for a supply medium to flow from the feed reservoir into the single-use bioreactor.

20. The component of claim 19, wherein the feed reservoir comprises a removable lid to allow the feed reservoir to be filled with a supply medium, and wherein the removable lid comprises a filter to allow air to replace dispensed supply medium.
